(19) United States
(12) Patent Application Publication
(10) Pub. No.: US 2003/0031647 A1
(43) Pub. Date: Feb. 13, 2003

(54) IFN-alpha and amantadine for treating hepatitis C

(76) Inventor: Friederike Zahm, Freiburg (DE)

Correspondence Address: HOFFMANN-LA ROCHE INC., PATENT LAW DEPARTMENT, 340 KINGSLAND STREET, NUTLEY, NJ, 07110

(21) Appl. No.: 10/236,268

(22) Filed: Sep. 6, 2002

Related U.S. Application Data
(63) Continuation of application No. 09/429,733, filed on Oct. 26, 1999.
Application No. 09/429,733 is a continuation of application No. 09/150,885, filed on Sep. 10, 1998, now abandoned.

(30) Foreign Application Priority Data
Sep. 18, 1997 (EP) 97116220.1

(51) Int. Cl. A61K 38/21; A61K 31/13

(52) U.S. Cl. 424/85.7; 514/662

(57) ABSTRACT

The present invention provides a method for treating chronic hepatitis C infections in patients in need of such treatment comprising administering an amount of IFN-α in association with an amount of amantadine effective to treat hepatitis C. In accordance with the invention, IFN-α and amantadine are administered at the same time or sequentially. The present invention also provides a kit for treating a hepatitis C infection containing IFN-α and amantadine in suitable dosage forms for administering the combination therapy.

### FIELD OF THE INVENTION

The present invention relates to the field of treatment of chronic hepatitis C infections using an amount of IFN-α in association with an amount of amantadine which amounts are effective to treat hepatitis C.

### BACKGROUND OF THE INVENTION

Interferons (IFNs) are naturally occurring proteins which have antiviral, antiproliferative and immunoregulatory activity. Four distinct classes of interferons are known to exist in humans (Pestka et al. (1987) Ann. Rev. Biochem. 56, 727-777 and Emanual & Pestka (1993) J. Biol. Chem. 268, 12565-12569). The IFN-α family represents the predominant class of IFNs produced by stimulated peripheral blood leukocytes (Pestka et al., loc. cit.; Havell et al. (1975) Proc. Natl. Acad. Sd. USA 72, 2185-2187; Cavalieri et al. (1977) Proc. Natl. Acad. Sd. USA 74, 3287-3291), and lymphoblastoid and myeloblastoid cell lines (Familletti et al. (1981) Antimicrob. Agents. Chemother. 20, 5-9). The antiviral effect of IFN-α is achieved not by a direct influence on the viruses themselves, but by an activity on their target cells in the sense of a protection against the virus infection. The interferons can exert effects on cancer tumors and can influence the immune system of the body in that, for example, they activate macrophages and NK cells and intensify the expression of various immunologically significant constituents of the cell membrane. Details of the preparation of interferon-cDNA and the direct expression thereof, especially in E. coli, have been the subject of many publications. Thus, for example, the preparation of recombinant interferons is known, for example, from Nature 295 (1982), 503-508, Nature 284 (1980), 326-320, Nature 290 (1981), 20-26, Nucleic Acids Res. 8 (1980), 4057-4074, as well as from European Patents Nos. 32134, 43980 and 211148.

IFN-α monotherapy is commonly used in the treatment of chronic hepatitis C infections, however this treatment is not always effective.

Amantadine has been proposed as monotherapy treatment for chronic hepatitis C infection (J P Smith et al., Treatment of chronic hepatitis C with amantadine hydrochloride, Abstract of the Annual Meeting of the American Gastroenterology Association, May 1996). However, this monotherapy treatment also does not result in response of all patients.

### SUMMARY OF THE INVENTION

The present invention provides a method for treating chronic hepatitis C infections in patients in need of such treatment comprising administering an amount of IFN-α in association with an amount of amantadine effective to treat chronic hepatitis C.

A combination of IFN-α and amantadine is provided which is more effective in treating hepatitis C infections than treatment with either IFN-α or amantadine alone.

In accordance with the present invention, IFN-α and amantadine can be administered separately and sequentially to treat hepatitis C infections. Alternatively, IFN-α and amantadine can be incorporated in effective amounts in pharmaceutical compositions for combined administration of the two compounds to treat hepatitis C infections.

### DETAILED DESCRIPTION OF THE INVENTION

The term “IFN-α” as used herein includes IFN-αs derived from any natural material (e.g., leukocytes, fibroblasts, lymphocytes) or material derived therefrom (e.g. cell lines), or those prepared with recombinant DNA technology. Details of the cloning of IFN-α and the direct expression thereof, especially in E. coli, have been the subject of many publications. The preparation of recombinant IFN-αs is known, for example from Goeddel et al. (1980) Nature 284, 316-320 and (1981), Nature 290, 20-26, and European Patents Nos. 32134, 43980 and 211148. There are many types of IFN-α such as IFN-αI, IFN-α2; and further their subtypes including but not limited to IFN-α2A, IFN-α2B, IFN-α2C and IFN-αII (also designated IFN-αII or ω-IFN). The term “IFN-α” also includes consensus IFN-α such as the consensus IFN-α described in U.S. Pat. No. 4,695,623, or mixtures of natural and/or recombinant IFN-αs. The use of IFN-α2A is preferred. The manufacture of IFN-α2A is described in European Patents Nos. 43980 and 211148.

The IFN-α used in this invention may be conjugated to a polymer such as a polyalkylene glycol (substituted or unsubstituted), for example poly-ethylene glycol, to form PEG-IFN-α. Conjugation may be accomplished by means of various linkers known in the art, particularly, by linkers such as those disclosed in European Patent Applications Publication Nos. 0510356 and 593868 (published on Oct. 28, 1992 and Apr. 27, 1994, respectively), and European Patent Application No. 97108261.5 (published on Dec. 3, 1997). The molecular weight of the polymer, which is preferably polyethylene glycol, may range from 300 to 70,000 daltons, and one or more, preferably one to three, polymers may be conjugated to the IFN-α. A preferred PEG-IFN-α conjugate has the formula:

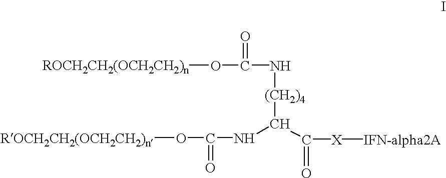

where R and R′ are lower alkyl; X is —NH—, and n and n′ are integers of from 400 to 550. The most preferred PEG-IFN-α conjugate is the compound of formula I wherein R and R′ are methyl, and n and n′ are not the same but one is 420 and the other is 520.

It is to be understood that in all instances where administration of IFN-α in association amantadine is described herein, PEG-IFN-α can be substituted for the IFN-α.

Amantadine, tricyclo[3.3.1.1^{3,7}]decane-1-amine, is described in the Merck Index, compound No. 373, Tenth Edition. Its manufacture is described in U.S. Pat. No. 3,152,180. In accordance with the present invention, the amantadine can be administered to a patient as amantadine, or a pharmaceutically acceptable salt thereof. Thus, it is to be understood that in all instances where administration of amantadine in association with IFN-α is described herein, the amantadine can be a pharmaceutically acceptable salt of amantadine. In the scope of the present invention, “pharmaceutically acceptable salts” include any salt chemically permissible in the art for amantadine and applicable to human patients in a pharmaceutically acceptable preparation. Any such conventional pharmaceutically acceptable salt of amantadine can be utilized. Among the conventional salts which can be utilized there are the base salts, including, for example, alkali metal salts such as the sodium or potassium salt, alkaline earth metal salts such as calcium or magnesium salt, and ammonium or alkyl ammonium salt. Preferred pharmaceutically acceptable salts of amantadine for use in the present invention are amantadine hydrochloride and amantadine sulphate.

In accordance with the present invention, administration of IFN-α in association with amantadine synergistically enhances the treatment provided by administering each ingredient separately. Any combined amounts of the two ingredients which will synergistically enhance the treatment provided by each ingredient can be utilized. The synergistic effect of the combined IFN-α and amantadine therapy results in a more effective reduction or disappearance of symptoms of hepatitis C infection. One exemplification of this enhanced synergistic effect is a greater reduction or disappearance of HCV RNA in peripheral blood mononuclear cells (PBMC) of chronic hepatitis C patients than with administration of either ingredient alone. To practice the invention, IFN-α and amantadine are administered to the patient suffering from chronic hepatitis C infection in amounts sufficient to provide more rapid elimination or greater alleviation of one or more of the signs or symptoms of chronic hepatitis C including elevated ALT, positive test for anti-HCV antibodies, presence of HCV as demonstrated by a positive test for HCV-RNA, clinical stigmata of chronic liver disease and hepatocellular damage than is achieved with administration of either IFN-α or amantadine alone.

The amantadine is administered to the patient in association with IFN-α, that is, the IFN-α dose is administered during the same or different periods of time that the patient receives doses of amantadine. At present, IFN-α formulations are not effective when administered orally, so the preferred method of administering the IFN-α is parenterally, preferably by subcutaneous (sc) or intramuscular (im) injection. The amantadine may be administered orally in capsule or tablet form in association with the parenteral administration of IFN-α. Of course other types of administration of both medicaments, as they become available are contemplated, such as by nasal spray, transdermally, by suppository, by sustainer release dosage form, etc. Any form of administration will work so long as the proper dosages are delivered without destroying the active ingredient.

The IFN-α can be administered parenterally in any amount which is effective to treat hepatitis C infections. Generally, however, the preferred dosage of IFN-α for practicing the combination therapy of this invention is about 1 to 6 million international units (lI) administered parenterally at least once weekly, preferably, twice or thrice weekly, every other day, or daily. The dosage may be administered according to any dosage schedule determined by the physician in accordance with the requirements of the patient. Also, dosage levels can be modified by the physician to be lower of higher than that stated herein depending on the needs of the patient, and the reaction of the patient to the treatment. The preferred dosage for practicing the combination therapy of this invention is about 3 million IU administered thrice weekly.

The amantadine can be administered orally in any amount which is effective to treat hepatitis C infections. A preferred oral unit dosage form comprises tablets, pills, sachets, or capsules of hard or soft gelatin, methylcellulose or of another suitable material easily dissolved in the digestive tract. Each tablet, pill, sachet or capsule can preferably contain from about 100 mg to 400 mg of active ingredient. The oral dosages contemplated in accordance with the present invention will vary in accordance with the needs of the individual patient as determined by the prescribing physician. The dosage levels can be modified by the physician to be lower of higher than that stated herein depending on the needs of the patient, and the reaction of the patient to the treatment. Generally, however, the dosage for oral administration of amantadine for practicing this invention is about 1.5 mg/kg to 6.5 mg/kg of body weight per day, preferably from about 3.0 mg/kg to about 4.0 mg/kg of body weight daily. The dosage may be administered according to any dosage schedule determined by the physician in accordance with the requirements of the patient. For example, the daily dosage may be administered once per day in a single dose or in divided doses twice or thrice per day. Preferably, the daily dosage is administered at least once weekly, and more preferably, one to three times weekly.

When IFN-α and amantadine are administered together in one composition, the preferred route of administration of the composition is parenteral administration, by either subcutaneous or intramuscular injection.

In accordance with the present invention, a kit for treating a hepatitis C infection is provided comprising a first component which is an IFN-α or a pegylated IFN-α in an injectable solution, the solution having an amount of IFN-α or PEG-IFN-α sufficient to administer to a patient in single or multiple injectable dosages of from about 1 million to about 6 million IU of IFN-α or PEG-IFN-α per dose. Preferably, the first component of the kit is a vial or a series of vials wherein the injectable solution in the vial contains an amount of IFN-α or PEG-IFN-α sufficient to provide a patient in a single administration of from about 3 million to about 4 million IU of IFN-α or PEG-IFN-α.

A second component of the kit contains amantadine or a pharmaceutically acceptable salt thereof in suitable oral unit dosage forms wherein each of the oral unit dosage forms contains from about 100 mg to about 400 mg of amantadine or a pharmaceutically acceptable salt thereof. Preferably, the second component includes amantadine or a pharmaceutically acceptable salt thereof in suitable oral unit dosage forms wherein each of the oral unit dosage forms contains from about 200 mg to about 300 mg of amantadine or a pharmaceutically acceptable salt thereof. The most-preferred oral unit dosage forms for the amantadine or pharmaceutically acceptable salt thereof are capsules or tablets.

The effectiveness of the treatment may be determined by controlled clinical trials of tars combination therapy versus monotherapy. The efficacy of the combination therapy in- alleviating the signs and symptoms of chronic hepatitis C infection and the frequency and severity of the side effects will be compared with previous IFN-α and amantadine monotherapy. Three populations suffering from chronic hepatitis C infection will be evaluated:

1. Patients previously untreated.

2. Patients previously treated with IFN-α or any other drug and who had subsequently relapsed.

3. Patients who were non-responsive to previous treatment with IFN-α or any other drug.

The effectiveness of the combination therapy is evaluated by determining the extent to which the previously described signs and symptoms of chronic hepatitis are alleviated.

### EXAMPLE

Antiviral Effect of Amantadine and IFN-α2A Against Hepatitis C Virus on Peripheral Blood Mononuclear Cells (PBMC) from Chronic Hepatitis C Patients

Mononuclear cells from patients with chronic hepatitis C (serum anti-HCV and HCV RNA positive with histologically proven chronic hepatitis) were analyzed for the presence of HCV RNA, using reverse transcription and PCR techniques with universal primers from the highly conserved 5′ non-coding region of the HCV genome (Navas et al., J. Hepatol. 21, 182-186 (1994)). Typing and subtyping of HCV genomes were performed by RFLP analysis of PCR products (Navas et al., J. Clin. Microbiol. 21, 317-321(1997)). For the purpose of this study, only cases infected by a single genotype have been considered, in order to minimize the possible interference of multiple genotypes, in the subject population, HCV subtype 1b was prevalent (Pernas et al., J. Gen. Virol. 76, 415-420(1995)). Thus, HCV RNA-positive PBMC obtained from 15 patients have been analyzed in vitro for the effects of treatment. PBMC from 10 matched healthy donors have been used as controls and analyzed similarly.

PBMC were isolated from heparinized venous blood by Ficoll-Hypaque gradient sedimentation. The interphase PBMC were isolated, washed twice with phosphate-buffered saline, and suspended in RPMI. The viability of these cells was assessed by trypan blue exclusion. PBMC were cultured in duplicate at a concentration of 2×10⁶ viable cells/ml in 6-well tissue culture dusters, in a humid atmosphere with 5% CO₂ for 7 days. The cultures have been maintained without mitogens (medium alone) or were stimulated with single mitogens (Phytohemagglutinin (PHA) or Lipopolysaccharide (LPS) or with PHA plus LPS (10 μg/ml each)(Martin et al., Cytokine 8, 313-317 (1996)). PBMC proliferation, and the possible drug-induced cytotoxicity, were measured using non-isotopic cell-proliferation and cytotoxicity assays.

The effect of experimental treatments with amantadine alone, in combination with IFN-α2A and those of IFN-α2A alone, have been established by testing HCV RNA in cultured mononuclear cells, compared with untreated PBMC from patients (Martin et al., supra); specificity controls were as described previously by Navas et al. in J. Hepatol. 21, 182-186 (1994). Treatment of mononuclear cells form healthy donors with amantadine alone, in combination with IFN-α2A or with IFN-α2A alone, served as controls. Changes in HCV RNA concentrations were measured by the AMPLICOR™ HCV MONITOR assay (Roche Diagnostic System, Inc., Branchburg).

Amantadine doses in the physiological range of 1-5 μM (2 μM corresponds to the therapeutically recommended blood level; daily dose of the drug: 100 mg/12 hours) did not affect the cell viability and had minor effects on the response to mitogens during cultures PBMC isolated from HCV patients and healthy donors. Higher doses of amantadine (50 and 500 μM were only investigated in PBMC from healthy donors. The dose of 50 μM slightly decreased PBMC proliferation, whereas the dose of 500 μM showed a marked anti-proliferative effect.

All PBMC cultures from HCV patients, but none from donors, were HCV RNA-positive with or without mitogens, as measured by a modification of the AMPLICOR™ HCV MONITOR assay. The dose of 2 μM amantadine reduced the mean amount of HCV RNA (number of copies/μg RNA) by >70% either alone and in combination with 1000 IU/ml IFN-α2A. In the individual patient, different degrees of reduction in HCV RNA concentration in PBMC were obtained after treatment with 1, 2 and 5 μM amantadine alone and in combination with 1000 IU/ml IFN-α2A (Table 1). In addition, HCV RNA became negative in up to 3/15 (20%) PBMC cultures (Table 1).

TABLE 1
Number of cases with reduction or disappearance of HCV RNA in
PBMC after experimental treatments (n = 15)

| Amantadine | IFN-α2A | Reduction in HCV RNA concentration | | | |
|---|---|---|---|---|---|
| (μM) | (IU/ml) | >25% | >50% | >75% | Negative |
| 1 | 0 | 3 | 2 | 3 | 0 |
| 2 | 0 | 5 | 2 | 4 | 1 |
| 5 | 0 | 2 | 2 | 3 | 3 |
| 0 | 1000 | 0 | 2 | 3 | 2 |
| 1 | 1000 | 3 | 3 | 4 | 0 |
| 2 | 1000 | 3 | 1 | 3 | 3 |
| 5 | 1000 | 0 | 0 | 2 | 3 |

HCV RNA became negative in PBMC cultures from 1/15 (7%) and 3/15 (20%) patients with the doses of 2 and 5 μM amantadine, respectively, compared with 2/15 (13%) with IFN-α2A alone. With the combination of amantadine and IFN-α2A 3/15 (20%) PBMC cultures resulted HCV RNA negative. The 2 μM amantadine/IFN-α2A combination had better results in the disappearance of HCV RNA in individual PBMC (up to 20% of cases; Table 1) showing a greater effect than the same doses of amantadine alone.

1. A method of treating hepatitis C in a patient in need of such treatment comprising-administering to said patient an amount of a first hepatitis C active ingredient in association with an amount of a second hepatitis C active ingredient wherein said first ingredient is an IFN-α or a pegylated IFN-α and said second ingredient is amantadine or a pharmaceutically acceptable salt thereof, said first and second ingredients being administered in combined amounts which synergistically enhance said treatment provided by administering each ingredient separately.

2. The method of claim 1, wherein the amount of the first ingredient is administered parenterally and the amount of the second ingredient is administered orally.

3. The method of claim 2, wherein the administration of the first ingredient is followed by administration of the second ingredient.

4. The method of claim 2, wherein the administration of the first ingredient is preceded by administration of the second ingredient.

5. The method of claim 2, wherein the amount of the first ingredient administered is from about 1 million IU to about 6 million IU at least once weekly.

6. The method of claim 5, wherein the amount of the first ingredient is administered from one to three times per week.

7. The method of claim 5, wherein the first ingredient is IFN-α2A.

8. The method of claim 5, wherein the first ingredient is PEG-IFN-α2A.

9. The method of claim 2, wherein the amount of the second ingredient administered is from about 1.5 mg/kg to about 6.5 mg/kg of body weight at least once weekly.

10. The method of claim 9, wherein the amount of the second 10 ingredient administered is from about 3.0 mg/kg to about 4.0 mg/kg of body weight at least once weekly.

11. A method of treating hepatitis C in a patient in need of such treatment comprising administering to said patient an amount of a first is hepatitis C active ingredient in association with an amount of a second hepatitis C active ingredient wherein said first ingredient is an IFN-α or a pegylated IFN-α and said second ingredient is amantadine or a pharmaceutically acceptable salt thereof, with the first ingredient being administered parenterally at from about 1 million IU to about 6 million IU at least once weekly and said second ingredient being administered orally at a dose from about 1.5 mg/kg to about 6.5 mg/kg of body weight at least once weekly.

12. The method of claim 11, wherein the first ingredient is IFN-α2A.

13. The method of claim 11, wherein the first ingredient is PEG-IFN-α2A.

14. The method of claim 11, wherein the first ingredient and second ingredient are each administered from one to three times weekly.

15. The method of claim 11, wherein the amount of the first ingredient administered is about 3 million IU to about 4 million IU one to three times weekly and the amount of the second ingredient administered is about 3.0 mg/kg to about 4.0 mg/kg of body weight one to three times weekly.

16. A kit for treating a hepatitis C infection said kit comprising a first component which is an IFN-α or a pegylated IFN-α in an injectable solution, said solution having an amount of IFN-α or PEG-IFN-α sufficient to administer to a patient in single or multiple injectable dosages of from about 1 million to about 6 million IU of IFN-α or PEG-IFN-α per dose, and a second component which is amantadine or a pharmaceutically acceptable salt thereof in suitable oral unit dosage forms, said oral unit dosage forms containing from about 100 mg to about 400 mg of amantadine or a pharmaceutically acceptable salt thereof.

17. The kit of claim 16, wherein the IFN-α is IFN-α2A.

18. The kit of claim 16, wherein the IFN-α is PEG-IFN-α2A.

19. The kit of claim 16, wherein said first component is a vial or a series of vials containing said injectable solution, said injectable solution in said vial being an amount of IFN-α or PEG-IFN-α sufficient to provide a patient in a single administration of from about 3 million to about 4 million IU of IFN-α or PEG-IFN-α.

20. The kit of claim 16, wherein the second component includes amantadine or a pharmaceutically acceptable salt thereof in oral unit dosage forms wherein each of the oral unit dosage forms in said second component contain from about 200 mg to about 300 mg of amantadine or a pharmaceutically acceptable salt thereof.

21. The kit of claim 16, wherein said second component includes amantadine or a pharmaceutically acceptable salt thereof in oral unit dosage forms wherein the oral unit dosage forms in said second component are capsules or tablets.

22. The method of claim 8, wherein the PEG-IFN-α2A is a conjugate of the formula: 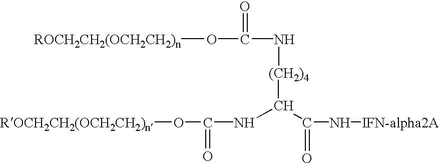
wherein R and R′ are lower alkyl; and n and n′ are integers of from 400 to 550.

23. The method of claim 22, wherein R and R′ are methyl.

24. The method of claim 22, wherein n and n′ are either 420 or 520 with the proviso that n and n′ are not the same.

25. The method of claim 13, wherein the PEG-IFN-α2A is a conjugate of the formula: 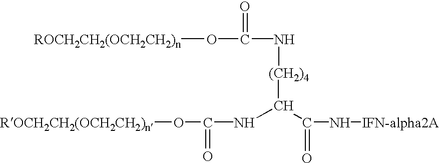
wherein R and R′ are lower alkyl; and n and n′ are integers of from 400 to 550.

26. The method of claim 25, wherein R and R′ are methyl.

27. The method of claim 25, wherein n and n′ are either 420 or 520 with the proviso that n and n′ are not the same.

28. The method of claim 18, wherein the PEG-IFN-α2A is a conjugate of the formula: 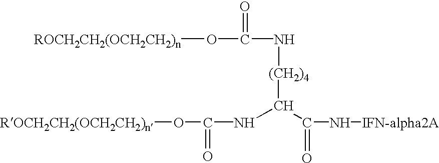
wherein R and R′ are lower alkyl; and n and n′ are integers of from 400 to 550.

29. The method of claim 28, wherein R and R′ are methyl.

30. The method of claim 28, wherein n and n′ are either 420 or 520 with the proviso that n and n′ are not the same.
